(19) United States
(12) Patent Application Publication
(10) Pub. No.: US 2026/0275579 A1
(43) Pub. Date: Sep. 17, 2026

(54) SYSTEM AND METHOD FOR MANUFACTURE OF COLLAGEN FIBERS

(71) Applicant: EMBODY, INC., Norfolk, VA (US)

(72) Inventors: Rohith Ramachandran Nair, Norfolk, VA (US); Amrita Dasgupta, Yorktown, VA (US); Nardos F. Sori, Virginia Beach, VA (US); Michael P. Francis, Norfolk, VA (US); Nathan Kemper, Madison, WI (US); Connor M. Donahue, Missoula, MT (US); Matthew B. Havener, Norfolk, VA (US)

(21) Appl. No.: 19/473,050

(22) PCT Filed: Apr. 5, 2024
(86) PCT No.: PCT/US2024/023353

§ 371 Date: Oct. 6, 2025

Related U.S. Application Data
(60) Provisional application No. 63/494,908, filed on Apr. 7, 2023.

(51) Int. Cl. D01D 7/00; D01D 1/02; D01D 5/04; D01F 4/00

(52) U.S. Cl. CPC D01D 7/00; D01D 1/02; D01D 5/04; D01F 4/00; D10B 2211/06; D10B 2509/00

(57) ABSTRACT

A method and system for producing biopolymer collagen fiber includes acidified collagen solution that can be piped to a vertical formation buffer bath. The collagen solution can be extruded as a stream of fibrous lines (filaments) after passing through a plurality of holes formed in a disc that can be disposed at the bottom end of the vertical formation buffer bath. Each filament can be immersed in the surrounding buffer bath and is polymerized, forming a collagen sub-filament. The resultant sub-filaments are extracted from the top of the vertical tank and gathered together into bundles to produce a collagen fiber.

### CLAIM OF PRIORITY

This application claims the benefit of U.S. Provisional Patent Application Ser. No. 63/494,908, filed on Apr. 7, 2023, the benefit of priority of which is claimed hereby, and which is incorporated by reference herein in its entirety.

### STATEMENT REGARDING GOVERNMENTAL SUPPORT

The data presented in this application was supported at least in part by DARPA SBIR 140D0420C0005. The US government has certain rights in the invention.

### BACKGROUND

### 1. Field of the Disclosure

The present disclosure relates to a method for manufacturing collagen fibers and their incorporation into scaffolds and implantable biocompatible devices prepared with such fibers. In particular, the disclosure relates to a method for extruding collagen fibers having superior mechanical strength, biocompatibility and immunological properties.

### 2. Description of Related Art

Collagen is a fibrous insoluble protein consisting of bundles of small reticular fibrils. Collagen fibrils combine to form white, glistening, inelastic fibers that are the primary component of connective tissues, including skin, bone, ligaments, and cartilage.

Many efforts have been made to manufacture collagen-containing scaffolds like tissue for use in the body to replace damaged collagen body parts, including in particular ligaments and tendons. Such implantable devices may replace the damaged part directly or may serve to provide a scaffold to facilitate repair of, and eventually replace, damaged soft tissues. Such products must function in a variety of challenging biomechanical environments in which multiple functional parameters must be addressed. These parameters include, for example, compatibility with bodily tissue and fluids, strength, flexibility, durability and biodegradability.

There is a need in the art for a system and method that addresses the shortcomings discussed above.

### SUMMARY

In one aspect, the present disclosure is directed to a method of producing a collagen biopolymer fiber. The method includes a first step of dissolving solid collagen in an acid solution to form a collagen solution, and a second step of extruding the collagen solution through a plurality of holes in a spinneret located at a bottom of a vertical formation buffer bath to form a plurality of sub-fibers. The method also includes a third step of bundling at least two sub-fibers of the plurality of sub-fibers together into a collagen fiber as the at least two sub-fibers ascend the vertical formation buffer bath. Furthermore, the plurality of sub-fibers and the resulting collagen fiber have a buoyancy such that they float from the bottom of the vertical formation buffer bath when extruded to the top of the vertical formation buffer bath.

In another aspect, the present disclosure is directed to a method of producing a collagen biopolymer fiber that includes a first step of connecting, via one or more tubes, a first container of collagen solution that is pumped into a spinneret, a second step of inserting the spinneret into a top opening of a vertical formation buffer bath, a third step of releasing the spinneret into the vertical formation buffer bath, a fourth step of extruding the collagen solution through a plurality of holes in the spinneret as the spinneret descends from the top opening to a bottom of the vertical formation buffer bath, and a fifth step of forming a plurality of sub-fibers.

In another aspect, the present disclosure is directed to an apparatus for producing a collagen biopolymer fiber. The apparatus can include a vertical formation buffer bath extending from a bottom end to a top opening, a spinneret disposed along the bottom end of the vertical formation bath, a container of acidified collagen solution, and a tubing system connecting the container of collagen solution to the spinneret.

Other systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like reference numerals designate corresponding parts throughout the different views.

FIG. 1 is a schematic flow diagram of an embodiment of producing a biopolymer collagen fiber;

FIG. 2 is a schematic view of an apparatus for manufacture of biopolymer collagen fibers, according to an embodiment;

FIG. 3 is a schematic view of an embodiment of a fiber formation system;

FIG. 4 is a schematic side view of an embodiment of a part of the fiber formation system;

FIGS. 5 and 6 are schematic perspective side views of a vertical formation buffer bath (tank) for forming collagen fibers, according to an embodiment;

FIG. 7 is a schematic view of a portion of a vertical formation buffer bath and accompanying spinneret, according to an embodiment;

FIG. 8 is a cross-sectional view providing an interior view of the spinneret of FIG. 7, according to an embodiment;

FIGS. 9A and 9B show a perspective top-down view of the collagen fibers collected from the vertical formation buffer bath as they are pulled into a horizontal formation buffer bath, according to an embodiment; and

FIG. 10 is a flow chart depicting a process of producing a biopolymer collagen fiber, according to an embodiment.

### DETAILED DESCRIPTION

The embodiments provide systems and methods for producing a biopolymer fiber. Biopolymer fiber is typically formed of collagen. In particular, atelocollagen or telocollagen typically is obtained from any source (human, bovine, recombinants, jelly fish, etc.). Bio-acceptable polymer, such as silk fibroin; other types of collagen such as type II collagen; fibrin/fibrinogen; basement membrane proteins; hyaluronic acid, poly ethylene oxide, poly ethylene glycol, poly caprolactone, polyethylene, polyhydroxybutyrate, PDLA; PDLLA and high molecular weight PDLLA; PLGA; and blends thereof, may be blended with collagen to form biopolymer fiber.

As described herein, collagen can be dissolved in an acid solution to form a collagen solution. In one embodiment, the prepared collagen solution is then passed or injected at a first speed through one or more apertures formed in a spinneret having a first diameter with a first speed. The spinneret is configured to enable the collagen solution to emerge from the slits as a plurality of slender filaments. The spinneret itself sits within a fiber-forming bath, such as a tank containing formation buffer. For purposes of this disclosure, the term “filament” (or fibrous line) refers to the acidified collagen solution that is being extruded but has not yet had sufficient exposure to the formation buffer to accumulate an exterior sheath and become a collagen fiber (or sub-fiber).

The formation buffer of the disclosed embodiments can be configured as an elongated vertical tank, and the emergence of the solution filaments from the apertures can be arranged to occur near or from the bottom of the vertical tank. Thus, as the filaments emerge, they are immersed in a reaction zone for a time and at speeds sufficient to form sheathed collagen sub-fibers, also referred to herein more simply “sub-fibers”. The greater buoyancy of the emerging filaments—quickly transforming into collagen sub-fibers—relative to the surrounding formation buffer causes them to naturally float upward toward the top of the vertical tank. These resultant sub-fibers can then be collected together at the top of the vertical tank and withdrawn. The sub-fibers may be collectively joined to form a single collagen fiber, which may be cross-linked and dried. In one example, they are withdrawn onto a spool at a speed between about 2 to about 10 times faster than the injection speed to increase molecular alignment and reduce the diameter of the fiber.

In various embodiments of the disclosure, collagen or collagen and other suitable biopolymers are made into biopolymer or collagen fiber. For ease of understanding, the features of the disclosure will be described as they relate to collagen. However, collagen may be blended or combined with suitable biopolymers in various combinations and proportions to obtain fibers of the type disclosed herein. In addition, throughout the specification, steps that might typically be taken together during a typical manufacturing process, such as washing and drying or soaking and drying, may be taken or repeated as appropriate to achieve a desired result. For example, in an embodiment, a composition may be washed and dried before advancing to the next step. In some embodiments, the material may be passed through a vertical tank of formation buffer a second time before advancing to the next processing step. In other embodiments, a first washing or drying step may be made optional. Thus, a material typically washed, then dried, may go directly to the drying step, and then moved on to the next processing step. The skilled practitioner can recognize circumstances under which steps may be repeated or eliminated.

In different embodiments, the constructs, such as scaffolds, made from the fibers, allow cellular ingrowth, that is, various types of cells from the animal tissue into which the fiber (and devices made from the fiber) is implanted will grow into the pores of the scaffold, preferably aligned with the fibers in the scaffold. Constructs and scaffolds comprise single layer and multi-layer articles that may be used as a substitute for a known repair feature, such as sutures used to re-attach body parts, for example opposing ends of a ruptured Achilles tendon. In addition to providing supporting structures for use in repairing torn or damaged tendons, embodiments of the disclosure are suitable in ligament repair as well. Thus, other exemplary ligaments for which the scaffolds or the present invention may be used to provide support include the ACL, MCL, PCL, UCL, and other human and animal ligaments. Other surgeries for which products of the disclosure are useful include superior capsular reconstruction as a treatment option for superior rotator cuff tears, and in particular for otherwise irreparable or difficult to repair partial or full tears. Similarly, a multi-layered sheet may be used to overlap a repair to strengthen it.

In particular, embodiments of the disclosure may be suitable for repair of ligaments, tendons, and other soft tissues of animals of all types. Collagen fibers of the disclosure may be used, for example, to reattach torn ligaments and tendons, even those with only a partial tear. Plural fibers also may be twisted, bundled, braided, interwoven, or otherwise arranged to improve a form factor that is easier to work with than a single fiber is to manipulate, for example during surgery. Improving the form factor may make it easier to locate a fiber or platform accurately. Other form factors may be constructed to serve as a reinforcement or internal brace for a torn natural body part. A brace connects from one bone to another bone to support a joint. Typically, a brace forms an isometric joint with restored biomechanics and the isometry of the native joint.

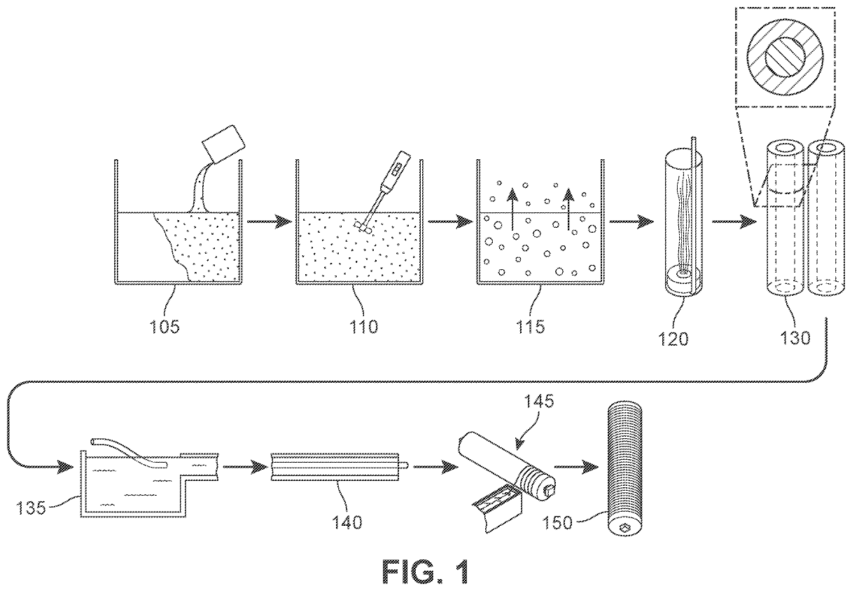

FIG. 1 illustrates an embodiment of a system and method for manufacturing collagen fiber. The system and method may be described as comprising four sections or manufacturing areas. A collagen solution can be prepared in the first section, and collagen fiber can be formed in the second section. The collagen fiber then is collected in the third section and then may be post-processed to yield wet or dry collagen fiber in the fourth section, post-treatments or end of treatment. The steps in the system and method illustrated in FIG. 1 may be grouped into four categories, as follows:

| Category | Name | Steps Included |
|---|---|---|
| 1 | Preparing Collagen Solution | 105-115 |
| 2 | Forming Collagen Fiber | 120-130 |
| 3 | Collecting Collagen Fiber | 135-150 |
| 4 | Post-Treatment or End Treatment | Not shown |

As seen at step 105 of FIG. 1, collagen can be combined with an acidic solution and stirred thoroughly at step 110. In some embodiments, the acid is between about 0.01 M and about 0.50 M acetic acid. In other embodiments, the acid is between about 0.01 M and about 0.50 M hydrochloric acid. The solution may be degassed at step 115. In some optional embodiments, the solution can also be centrifuged, for example to help remove residual bubbles.

Resultant collagen solution can be then injected into a bath of formation solution to form a plurality of sub-fibers in step 120. The resultant forming sub-fibers may incorporate a coaxial sheath (shown in cross-section surrounding or encasing the extruded solution filament) in step 130. This resultant product is a formed collagen sub-fiber, which can be joined together to produce a collagen fiber. In different embodiments, the fiber then continues to a collection system, wherein the fiber is separated from the formation buffer solution at step 135, passed through an ethanol bath, and dehydrated at step 140. The ethanol bath increases the strength of the fiber. The collagen fiber is recovered at step 145 and collected on a spool and air-dried at step 150. Then, post-processing may be carried out. Some examples of these techniques are described in further detail in U.S. Pat. No. 11,020,509 issued on Jun. 1, 2021 and titled “Microfluidic Extrusion” to Francis, et al. (hereinafter the Francis application), the disclosure of which is incorporated by reference herein in its entirety.

FIG. 1 is intended to provide a generalized view of a system and method for carrying out an embodiment of the disclosure. Additional details and disclosure are included in the following particular aspects and embodiments of the description below.

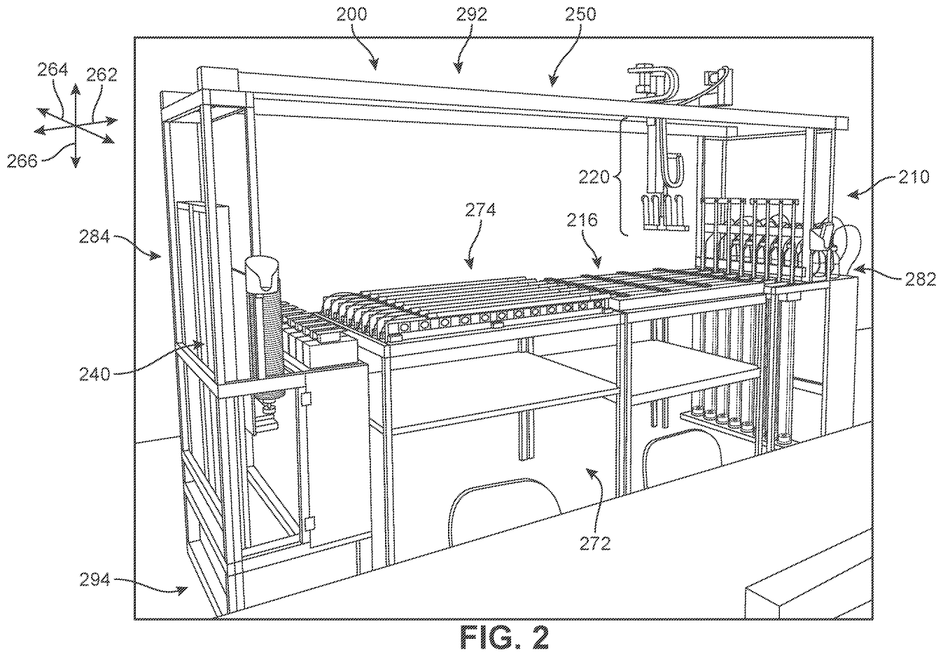

FIG. 2 introduces an embodiment of a collagen fiber manufacturing apparatus (“apparatus”) 200. In different embodiments, the apparatus 200 can be understood to include multiple interconnected systems, including a fiber formation system 210, a fiber pulley system 220, an alcohol immersion bath system 216, a fiber drying system 230, and a fiber collection system 240. In one example, the systems are stabilized and held together by a framework 250. Suitable arrangements and operations of the fiber pulley system 220 and the alcohol immersion bath system 216 are described in (172-MBDY-038), the entire disclosure of which is herein incorporated by reference. Furthermore, suitable forced air drying box sections are described in (172-MBDY-039), the entire disclosure of which is herein incorporated by reference. The following description will primarily discuss aspects and features related to the fiber formation system 210.

For clarity, the description makes reference to distal and proximal directions (or portions) in the context of the larger apparatus 200. As used herein, the distal direction is a direction oriented away from the fiber formation system 210 and toward the fiber collection system 240, while the proximal direction is a direction oriented toward the fiber formation system 210 and away from the fiber collection system 240. The proximal and distal directions can also be understood to refer to opposing directions relative to a longitudinal axis 262 characterized in FIG. 2. Thus, the term “longitudinal” as used throughout this detailed description and in the claims refers to a direction extending between a proximal side 292 and a distal side 294 of the apparatus 200. For example, the first horizontal bath 342 has a length aligned with the longitudinal axis 262 that extends from the direction of the anchor portions in a proximal direction.

Also, the term “lateral” as used throughout this detailed description and in the claims refers to a direction extending along a width of a component/system. In other words, the lateral direction may extend between a medial side 272 and a lateral side 274 of the apparatus 200, characterized by a lateral axis 264 in FIG. 2. Furthermore, the term “vertical” as used throughout this detailed description and in the claims refers to a direction generally perpendicular to a lateral and longitudinal direction. This is characterized by a vertical axis 266 in FIG. 2 that extends in a direction between a top side 292 and a bottom side 294 of the apparatus 200. For example, in cases where a component has an end nearer a ground surface, the vertical direction may extend from the ground surface upward, such as along the length of each of the vertical tanks. Thus, each axis of the three axes may be understood to be orthogonal relative to the other two axes. Additionally, the term “inner” refers to a portion of a component disposed or enclosed by an outer surface, such as the interior chamber of the vertical tanks which hold the buffer. Likewise, the term “outer” refers to a portion of a component disposed further from the interior.

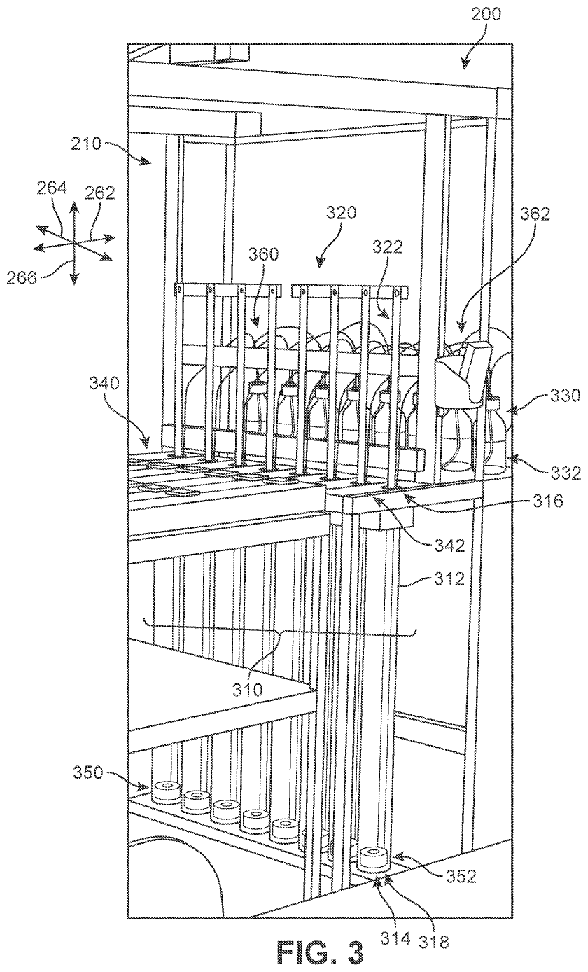

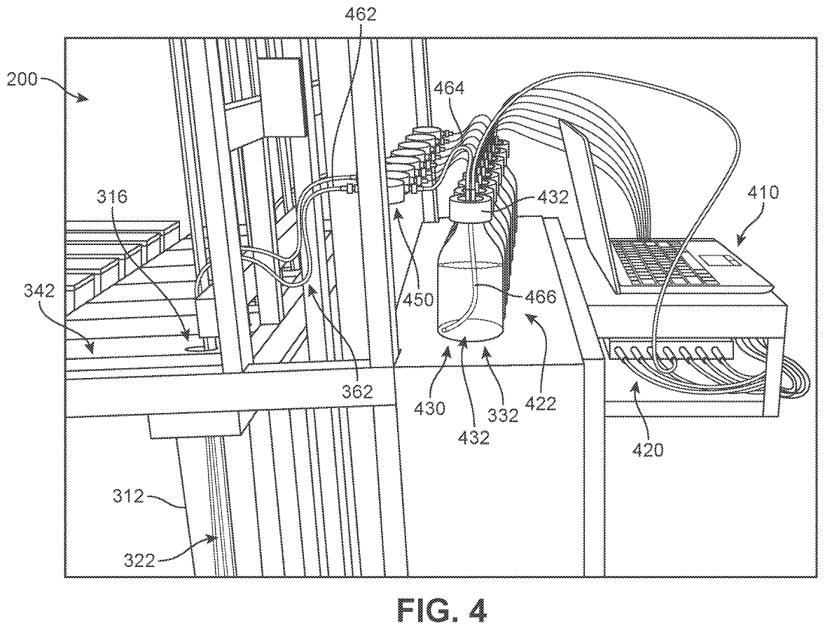

For purposes of clarity, FIG. 3 depicts an isolated view of an embodiment of the fiber formation system 210. In this example, fiber formation system 210 includes a series of components, in this case including a plurality of vertical tanks 310, a plurality of horizontal baths 340, a plurality of containers 330 in which collagen solution can be held, an optional plurality of anchor portions 320, a plurality of spinnerets 350, a plurality of tubing elements 360, a plurality of flow meters (depicted in FIG. 4), a computing device (depicted in FIG. 4) and a pressure controller device (depicted in FIG. 4). It can be observed that the apparatus 200 include a repeating arrangement of components. In other words, the fiber formation system 210 can also be understood to comprise multiple substantially identical production assemblies, where each production assembly includes one vertical tank, one horizontal bath, one container, one optional anchor portion, one spinneret, one flow meter, and one set of tubing elements, which can be in communication with the pressure controller device and the computing device. The apparatus 200, by incorporating a greater number of assemblies, allows for mass production of the collagen fibers, as well as the maintenance of a continuity of production in the case of errors or malfunction during operation of one of the assemblies.

For purposes of simplicity, the discussion herein will focus on a single production assembly of the fiber formation system 210, with the understanding that there may multiple such assemblies working in concert, or side by side, in apparatus 200, as illustrated in FIG. 2. In FIG. 3, a first assembly thereby can be understood to include a first vertical tank 312, also referred to herein as vertical formation buffer bath that extends from a topmost first end 316 to a bottommost second end 314. In some embodiments, the first vertical tank 312 can be mounted or secured along a backbone or framework (e.g., a first anchor portion 322) that helps stabilize the tank. The first assembly of the fiber formation system 210 further includes a first spinneret 352, shown in FIG. 3 as disposed within the first vertical tank 312. The term “filament extrusion facilitation device” or simply “extruder” can also be used in lieu of the term spinneret. In one embodiment, the first spinneret 352 can be disposed directly above or atop a base portion 318. In one embodiment, the base portion 318 comprises a substantially continuous (one-piece) component that extends beneath and supports each of the plurality of tanks. The base portion 318 can be in contact with the bottommost second end 314.

In different embodiments, the first assembly also includes a first container 332, such as a beaker or flask or other container configured to hold liquid, in which a reservoir of collagen solution can be stored and secured. The fiber formation system 210 also includes a first tubing subsystem 362 comprising one or more elongated tubes that connect the contents of the first container 332 with the interior of first vertical tank 312, via first spinneret 352. An optional first horizontal bath 342 extends from or can be directly adjacent to a first anchor portion 322, where the topmost first end 316 of the first vertical tank 312 has an opening that allows the contents of the first vertical tank 312 to be in fluid communication with the contents of the first horizontal bath 342, providing an upside-down and inverted L-shaped fluid storage arrangement.

For purposes of clarity to the reader, the embodiments may be characterized by various directional adjectives and reference portions. These directions and reference portions may facilitate in describing the portions of a system, components thereof, and/or the apparatus as a whole. Moreover, these directions and reference portions may also be used in describing each assembly of the apparatus (e.g., devices, mechanical components, and other structural features). Thus, for consistency and convenience, directional adjectives are employed throughout this detailed description corresponding to the illustrated embodiments.

Moving now to FIG. 4, a closer view of the components of the first assembly of apparatus 200 is provided. In different embodiments, aspects and operations the fiber formation process can be managed and/or monitored via a computing device 410. Computing device 410 can include provisions for communicating with, and processing information from, each of the components of apparatus 200, as well as a controller and other devices. The computing device 410 may include one or more processors and memory. Memory may comprise a non-transitory computer readable medium. Instructions stored within memory may be executed by the one or more processors. In addition, computing device 410 may include a communication system such as a radio or other provisions for communicating using one or more communication methods. In particular, communication system includes provisions for communicating with other nearby devices and/or platform 250 over networks 202. For example, each communication system could include a Wi-Fi radio, a Bluetooth radio, and/or a cellular network radio.

The processes and methods of the embodiments described in this detailed description and shown in the figures can be implemented using any kind of computing system having one or more central processing units (CPUs) and/or graphics processing units (GPUs). The processes and methods of the embodiments could also be implemented using special purpose circuitry such as an application specific integrated circuit (ASIC). The processes and methods of the embodiments may also be implemented on computing systems including read only memory (ROM) and/or random access memory (RAM), which may be connected to one or more processing units. Examples of computing systems and devices include, but are not limited to: servers, cellular phones, smart phones, tablet computers, notebook computers, e-book readers, laptop or desktop computers, all-in-one computers, as well as various kinds of digital media players.

The processes and methods of the embodiments can be stored as instructions and/or data on non-transitory computer-readable media. The non-transitory computer readable medium may include any suitable computer readable medium, such as a memory, such as RAM, ROM, flash memory, or any other type of memory known in the art. In some embodiments, the non-transitory computer readable medium may include, for example, an electronic storage device, a magnetic storage device, an optical storage device, an electromagnetic storage device, a semiconductor storage device, or any suitable combination of such devices. More specific examples of the non-transitory computer readable medium may include a portable computer diskette, a floppy disk, a hard disk, magnetic disks or tapes, a read-only memory (ROM), a random access memory (RAM), a static random access memory (SRAM), a portable compact disc read-only memory (CD-ROM), an erasable programmable read-only memory (EPROM or Flash memory), electrically erasable programmable read-only memories (EEPROM), a digital versatile disk (DVD and DVD-ROM), a memory stick, other kinds of solid state drives, and any suitable combination of these exemplary media. A non-transitory computer readable medium, as used herein, is not to be construed as being transitory signals, such as radio waves or other freely propagating electromagnetic waves, electromagnetic waves propagating through a waveguide or other transmission media (e.g., light pulses passing through a fiber-optic cable), or electrical signals transmitted through a wire.

Instructions stored on the non-transitory computer readable medium for carrying out operations of the present invention may be instruction-set-architecture (ISA) instructions, assembler instructions, machine instructions, machine dependent instructions, microcode, firmware instructions, configuration data for integrated circuitry, state-setting data, or source code or object code written in any of one or more programming languages, including an object oriented programming language such as Smalltalk, C++, or suitable language, and procedural programming languages, such as the “C” programming language or similar programming languages.

As described earlier, in some embodiments, a collagen solution 430 can be stored or otherwise held in first container 332. In different embodiments, the first assembly includes provisions for transporting or moving the fluid in the first container 332 to the first vertical tank 312. An example of this arrangement includes the elements of the first tubing subsystem 362, which for purposes of reference can include a first tube portion 466, a second tube portion 464, and a third tube portion 462. The tube portions can represent the same tube or multiple pieces of tubes. The first tube portion 466 has an open end that can be submerged in the collagen solution 430 in first container 332. The first tube portion 466 extends upward toward the first container 332, and exits the first container 332 through an optional cap portion 432. The first tubing subsystem 362 then continues toward a flow meter device 450 via second tube portion 464. In some embodiments, the first tube portion 466 and the second tube portion 464 are segments of the same continuous tube, while in other embodiments, the two portions are separate but connected to one another through a connector element.

The second tube portion 464 can be mated to a connector tab of the flow meter device 450, which regulates the flow rate in conjunction with a pressure control device (“pressure controller”) 420. It should be understood that the pressure controller 420 can include closed loop feedback control. In some embodiments, the pressure controller 420 can include a syringe or syringe pump, and/or programmable pumps, pressure-based flow controllers, microfluide flow controls, and other available or standard pressure control devices.

The pressure controller 420 can include a pressure connector tubing 422 that can be connected to the first container 332 and controls the pressure in the first container 332. In different embodiments, the pressure controller 420 provides positive pressure within the first container 332 to allow for the withdrawal of the collagen solution via the tubing subsystem. Through this arrangement, the collagen solution 430 can be drawn up into the first tube portion 466, into the second tube portion 464, through a proximal side of the flow meter device 450, and out the distal side of the flow meter device into the third tube portion 462 at a measured, steady, and/or consistent rate.

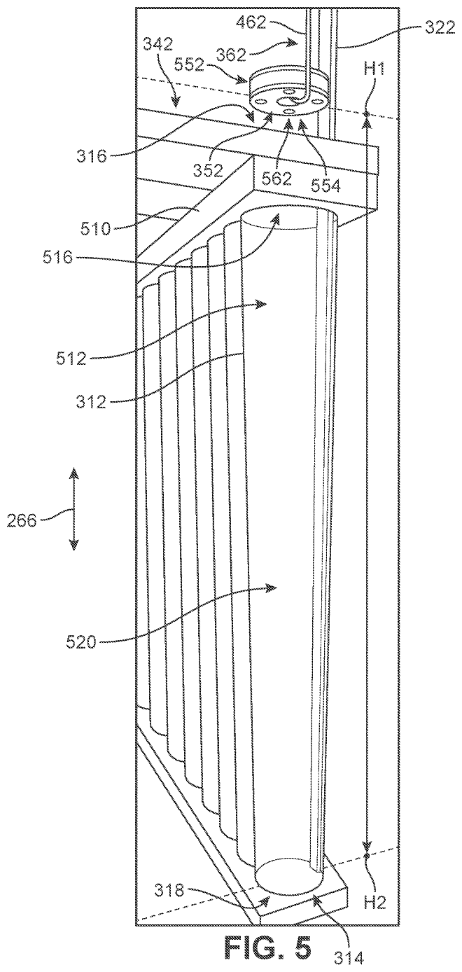

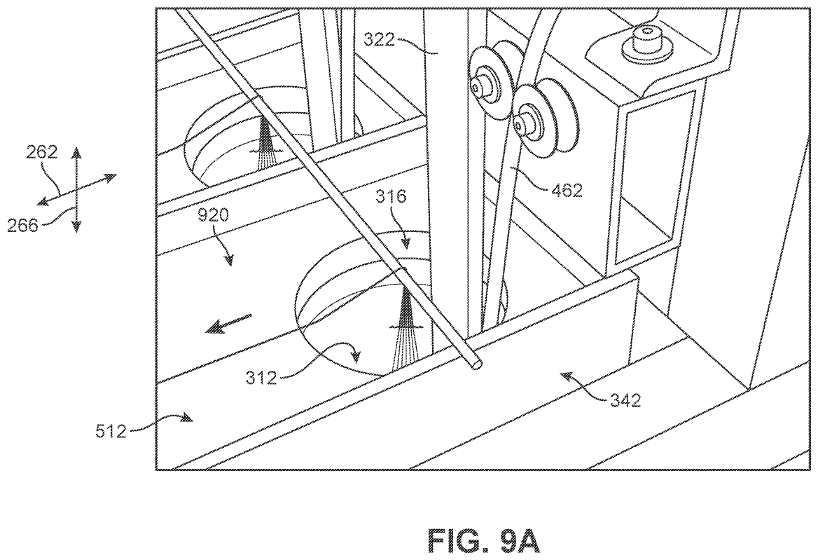

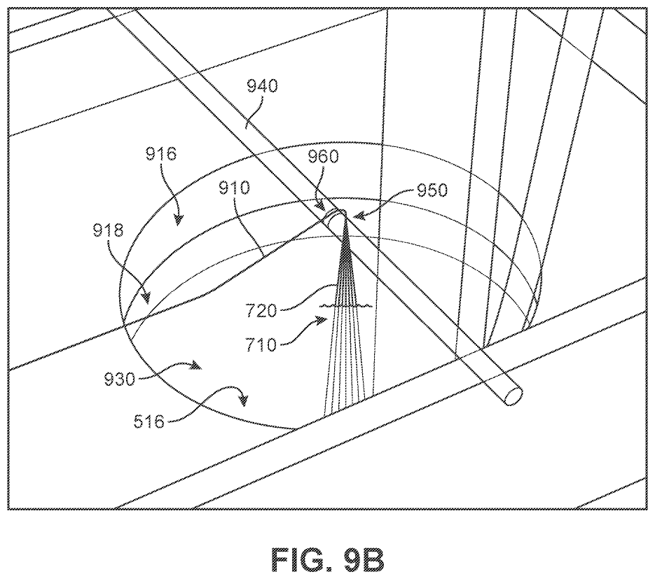

Referring now to FIG. 5, in some embodiments, the distal end/outlet of the third tube portion 462, representing a distal end of the first tubing subsystem 362 can be connected to the first spinneret 352, as will be discussed in greater detail below. Prior to and during an initial loading stage, as the tubing can be initially connected to the first spinneret 352, the first spinneret 352 can be disposed outside of and above the first end 316 of the first vertical tank 312. This is better seen in FIG. 5, which depicts a schematic view of the first vertical tank 312 and first spinneret 352 in its initial loading configuration. In some embodiments, the vertical tank includes a mating panel 510 which can help securely fasten or join to a corresponding opening in the first horizontal bath 342 (e.g., see FIGS. 9A and 9B), allowing the liquid (i.e., buffer 512) held within the compartments of both the first horizontal bath 342 and the first vertical tank 312 to form a seal, providing fluid communication between the two components.

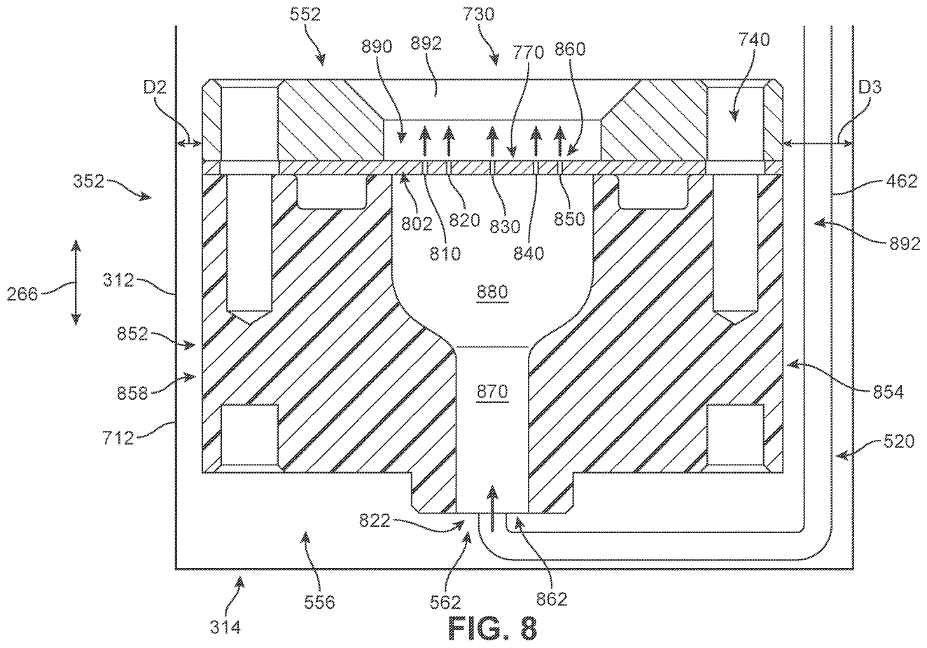

Thus, in some embodiments, during the loading stage, the first spinneret 352 can be disposed directly above both the opening 516 in the first vertical tank 312, as well as the opening in the first horizontal bath 342, which are stacked or sealed together vertically. A topside 552 of the first spinneret 352 faces directly upward, and an opposite underside 554 of the first spinneret 352 faces directly downward toward the base portion 318 that sits underneath the first vertical tank 312. The end of the third tube portion 462 can be secured to a port provided along the underside 554 of the first spinneret 352 (e.g., see FIG. 8). Because the third tube portion 462 is connected beneath the first spinneret 352, it then extends out and is oriented such that the tube then travels away and upward back toward the first container. In this initial loading configuration, the first spinneret 352 is at a first height H1 above the base portion 318, which is outside of the vertical tank 312 and directly above an opening 516 at the first end 316 that provides access to the interior volume contained by the vertically aligned exterior housing or surface vertical tank 312. In different embodiments, the opening 516 is substantially round, and the vertical tank 312 has a substantially circular cross-sectional shape along the horizontal plane and a substantially cylindrical three-dimensional shape. In some embodiments, the shape of the vertical tank 312 can be selected to match or align with the shape of the first spinneret 352, which in this case is substantially cylindrical and squat, akin to a puck or a disc. Thus, in other embodiments, the shapes of the spinneret and the vertical tank can differ, but remain aligned or match.

Once the first spinneret 352 has been securely connected to or mated with the open end of the third tube portion 462, fluid can flow into the first spinneret 352 from the opposite end of the first tubing subsystem 362 (e.g., the collagen solution in the first container) and the loading phase is complete. A deployment stage can next occur whereby a collagen dispersal assembly, comprising the first spinneret 352, the first tubing subsystem 362, and the collagen solution flowing through the first tubing subsystem 362, can be released. In one example, the first height H1 is approximately 0.5 inches to 1.5 inches above the accompanying opening in the horizontal bath before being dropped, though in other embodiments, the first height H1 can be closer to the surface of the buffer fluid and the passageway leading into the interior of the vertical tank. The collagen dispersal assembly drops or falls into an interior chamber 520 of the first vertical tank 312 via opening 516. The first spinneret 352 has a density that allows it to readily sink down the length of the first vertical tank 312.

In other embodiments, an optional loading device (also referred to herein as a “guide device”) that grips or removably attaches to the first spinneret can be used to gently guide the spinneret to the bottom and then releases the spinneret. For example, the spinneret can be disposed on or rest on a fixture that can be connected to the guide device, such as a plate or other support. The fixture can include mating pins or other fastening elements that, when secured to the spinneret, help prevent misalignment of the spinneret throughout the extrusion process. In some embodiments, the guide device can be used to gently move the spinneret down the length of the vertical tank at a set speed which can be the same or approximately equal to the designated fiber collection speed. In one example, the guide device can be removed or pulled back out of the vertical tank at the end of the extrusion session. In some embodiments, the guide device stays in place once the extrusion process begins to help maintain a fixed fiber draw start position.

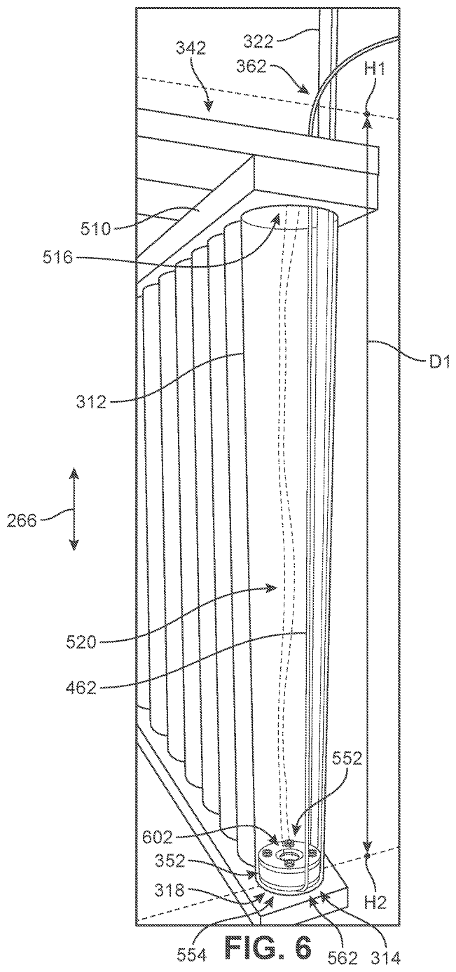

In some embodiments, the outermost circumference of the first spinneret 352 can be slightly smaller than the outermost circumference of the opening 516, to allow for passage of the first spinneret 352 when it is deposited into the vertical tank 312 (see FIG. 6). The guide device and its associated fixture allow the spinneret to travel through the tube while preventing the first spinneret 352 from rotating (i.e., thereby maintaining the orientation of the topside 552 and the underside 554 relative to the vertical axis 226).

Referring next to FIG. 6, as the collagen dispersal assembly sinks a first distance D1, from the first height H1 to the second height H2, collagen solution can flow or extrude from a plurality of apertures formed in a central core of the first spinneret 352 (e.g., see FIG. 8). In other words, as the collagen solution is fed to the first spinneret 352 from the first container via the first tubing subsystem, it can exit out of tiny through-holes formed in the spinneret. As a portion of the underside 554 contacts and rests securely against the bottommost second end 314 directly atop the base portion 318, along with a bent or curved portion of the third tubing portion 462 attached to the first spinneret 352, the deployment stage can be concluded.

In some embodiments, during the fall or drop of the first spinneret 352 down the tube, a substantially steady stream of viscous collagen solution 602 can be emitted or extruded from the first spinneret 352 in a microfluidic state (as filaments) that has a fibrous tendril or thread-like configuration at a substantially steady or constant rate, signaling the beginning of a collagen formation stage. In general, in different embodiments, the first distance D1 can be selected to maximize immersion time and travel of the solution up the vertical tank, with the understanding that a taller (longer) vertical tank and buffer immersion experience will cause the sub-fibers to increase in strength correspondingly. However, the first distance D1 should not exceed the threshold whereby the weight of each sheathed sub-fiber—increasing as they float upwards due to the continuously growing sheath around the core (e.g., the extruded solution filament)—neutralizes or overcomes the natural buoyancy of the collagen solution, in order to prevent the sub-fibers from sinking before reaching the top end of the vertical tank. In other words, while the collagen fiber enters the tank as buoyant and less dense than the formation buffer fluid, as the fiber floats upwards, water can be removed from the fiber as it crosslinks, increasing the collagen fiber's density. Eventually, if the vertical buffer tank is too tall, the density of the collagen fiber becomes greater than the formation buffer fluid and the collagen fiber begins to slowly sink. The maximum preferred height of the vertical buffer tank should therefore be selected to fall below this distance. If the vertical buffer tank exceeds this height, the fibers would begin to sink, making the fibers difficult to collect. Thus, in different embodiments, a height of the vertical buffer tank may be restricted to ensure that the density of the collagen fiber does not become greater than that of the formation buffer fluid as it travels up the tank over the first distance D1.

In different embodiments, the initial release of the strands or fibrous lines of collagen solution can be arranged to occur during the deployment, e.g., while the collagen dispersal assembly falls down into the vertical tank. For example, as shown in FIG. 6, the collagen solution 602 can be pumped and pushes through channels in the spinneret as the spinneret is moving downward. In some embodiments, the immersion rate of the assembly is substantially constant. In one example, the immersion rate can be approximately matched to the pump rate of the collagen solution from the topside of the spinneret, allowing for a “cast-in-place” type process of the filaments as the spinneret falls. As described earlier, each of the extruded filaments are buoyant relative to the surrounding buffer. Thus, as the spinneret falls, the filaments (fibrous lines) exiting the spinneret will float upwards, transforming into sub-fibers of increasing strength along their journey to the top. As each of the fibrous lines of acidified collagen exit from the top end of each aperture in the first spinneret 352 (see FIG. 8), they flow into a reaction zone comprising a fibril-forming bath, or a formation buffer solution, which will cause formation of a sheath around each collagen sub-fiber, and begins to form as a solid sub-fiber. These sub-fibers continue to rise upward toward the top of the vertical tank.

In different embodiments, it can be appreciated that the extrusion process should be initiated from a top-down drop of the collagen dispersal assembly in order to overcome the hydrostatic pressure that could otherwise prevent or hinder the initial extrusion and upward drift of the individual sub-fibers. In other words, if the extrusion were to be delayed until the spinneret had reached the bottom of the vertical tank, the buffer could potentially ‘back-up’ into the tubing and undesirable formation/sheathing may occur prematurely or haphazardly upstream in the tube itself, rather than in the vertical tank. In one example, the buffer would then block the apertures via which the filaments are to be extruded. Thus, the initial drop deployment and simultaneous casting/extruding as the spinneret falls downward can be an important and necessary aspect of the formation process.

It should be appreciated that the vertical extrusion described herein can be configured to help maintain uniform tension in each of the individual fibers until the strand has been formed at the top. In addition, both filament contact area and filament contact time with the surrounding formation buffer can be greater when compared to similar immersion in a horizontal, rather than a vertical, tank. This increase in filament contact area and filament contact time can increase fiber strength (e.g., breaking load), which will later facilitate the feasibility of the braiding process. In addition, vertical extrusion (compared to horizontal extrusion) promotes a circular shape of the fiber, rather than production of a fiber with a flat/ribbon-shape profile.

In one example, formation buffer solution can be used to neutralize the collagen solution and to assist with fibrillogenesis. In different embodiments, the coaxially-flowing collagen fibrous lines (filaments) pass through a reaction zone comprising a fibril-forming bath for a time and at volumetric flow rates sufficient to form a sheathed fiber with a strength described herein. In some embodiments, formation buffer solution provided in the vertical tanks may be any solution that aids formation of a collagen fiber. Formation buffer solution typically can be a solution comprising of various salts and buffering agents. In some embodiments of the disclosure, formation buffer solution can be WSB, a solution comprising 6.85 g/L TES, 4.14 g/L sodium phosphate monobasic dihydrate, 12.1 g/L sodium phosphate dibasic, 7.89 g/L NaCl, and 10 percent w/v PEG (polyethylene glycol) and of pH greater than or equal to 8. Similar solutions also may be suitable.

In different embodiments, the flow rates of the collagen solution as it passes upward as sub-fibers along the vertical tank relative to the surrounding formation buffer solution are adjusted so as to provide a stretch to the collagen fiber and improve the quality of the fiber. Indeed, in this way, the collagen will be urged to form a relatively straight, continuous fiber without kinks and other physical shape aberrations. In some embodiments, fibers may be substantially circular, ovoid, square, rectangular, ribbon-like, triangular, or irregularly shaped. In some embodiments, the relative speed of the collagen fibers as they float upwards as well as the pull of the collecting spool (e.g., see FIG. 9) can be used to pull or stretch the collagen stream, which creates an extensional field that helps align the collagen monomers in a process called flow-induced crystallization. This alignment helps collagen polymerize and increases the strength of the resultant product.

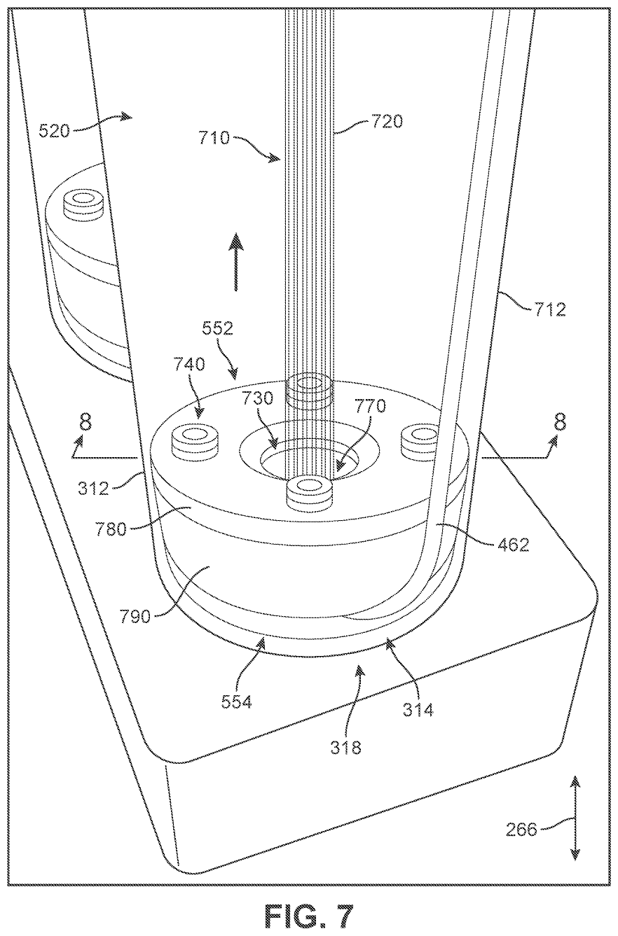

As noted above, during the collagen formation stage, the filaments are extruded at a substantially steady rate and then immersed in the buffer provided in the vertical tank. For purposes of clarity to the reader, FIGS. 7 and 8 provide additional details regarding the spinneret and extrusion process. In FIG. 7, a lower region of the first vertical tank 312 atop the base portion 318 is illustrated. The first vertical tank 312 has a continuous outer housing 712 extending from the top end to the bottom end that encloses a watertight interior volume (chamber) that in this case holds the formation buffer. In this example, the outer housing 712 is clear (e.g., plexiglass) to better reveal the interior activity for the reader. However, in other embodiments, the outer housing 712 can be translucent or opaque. FIG. 7 depicts the first spinneret 352 resting or disposed above the base portion 318, against or touching the bottommost second end 314 of the interior of the first vertical tank 312. The third tube portion 462 can be seen as it exits from underneath the underside 554 of the first spinneret 352 and can be routed upward along the interior chamber of the first vertical tank 312.

In different embodiments, the first spinneret 352 can include a recessed area 730 in a central area of its topside 552. The center of the recessed area 730 (the lowest part of the dip) will be referred to as extrusion zone 770. The extrusion zone 770 includes a substantially solid or continuous surface, but for a plurality of through-hole apertures (e.g., see FIG. 8) formed in the surface, also referred to herein as a “plurality of holes”. From each of these apertures, a plurality or stream of solution extruded collagen fibrous lines (“filaments”) 710, including a first filament 720, are being extruded and immersed in the buffer formation bath. As they are extruded, each of the filaments 710 have a natural buoyancy that causes them to float upward. During their journey upward, with the steady exposure to the surrounding buffer formation bath, each filament will become increasingly ‘sheathed’, thereby transforming into a collagen sub-fiber. The farther the sub-fibers travel in the buffer formation bath, the greater the exposure to the buffer, and the stronger the resultant sub-fibers become. In some embodiments, the first spinneret 352 can also include mechanical features, such as connector mechanisms 740, that secure a lid portion 780 to a body portion 790. The recessed area 730 can be provided in the lid portion 780, and includes an outer wall (see extrusion perimeter wall 890 in FIG. 8) that encircles or forms a perimeter about the extrusion zone 770, helping to guide the extruded fibrous lines upward, while the main extrusion components are within the body portion 790. Thus, before the collagen exits the spinneret, it is in its solution form, and once it leaves the spinneret, it takes on an extruded form (filament or fibrous line) that will be encased to form the collagen sub-fiber.

Further details regarding the structure of the spinneret are depicted in a cross-sectional view of the first spinneret 352 in FIG. 8, taken along the line 8-8 in FIG. 7, and extending across the width of the tank. In FIG. 8, the third tube portion 462 can be more clearly seen as it extends downward alongside a proximal side 854 of the first spinneret 352, curving inward and terminating at a spinneret entry port 862. An outlet 822 of the third tube portion 462 can be connected or mated to the entry port 862, forming an airtight seal. As collagen solution 892 is pumped down through the tubing, it can be passed into the entry port 862, and travels upward through a receiving conduit 870, via which the collagen solution 892 can be pushed along to a wider dispersal chamber 880, where it spreads up and out until arriving at an extrusion plate 802. The extrusion plate 802 can be directly under and adjacent to the extrusion zone 770 and can be understood to include the opposite-facing side of the extrusion zone 770. A plurality of through-hole apertures (“apertures”) 860 are formed within and extend the full thickness of the extrusion plate 802 and form a set of vertical channels, including a first channel 810, a second channel 820, a third channel 830, a fourth channel 840, and a fifth channel 850. Each channel includes an inlet or first opening at its lower end, in fluid communication with the dispersal chamber 880, and an outlet or second opening at its upper end, in fluid communication with the contents of the vertical tank. Each channel can be sized and dimensioned to allow for the upward extrusion of microfluidic filaments of the collagen solution. As the filaments exit each of the apertures 860, they pass through the space in the lid portion 780 (see FIG. 7) bounded by the extrusion perimeter wall 890 and wider and sloped outer 892 wall that together surround the extrusion zone 770 and guide the filaments up and away from the spinneret.

As a general matter, the internal chamber of first spinneret 352 in the base portion can be configured such that the fibers passing through the channels are extruded in essentially equal quantities. In different embodiments, the number of apertures (channels) provided can be understood to approximately equal the number of fibrous lines that will be extruded and sheathed simultaneously. In this example, 26 apertures corresponding to the outlets for 26 through-hole channels allow for simultaneous production of 26 collagen fibers. In some other embodiments, the spinneret may have more or fewer channels formed within its body. As one non-limiting example, collagen solution can be passed through the tubing measuring approximately 3.175 mm or ⅛ inch in diameter into the entry port formed in the spinneret housing. The entry port cavity then expands to a larger size in the entry conduit, for example to approximately 15 mm in diameter. In some embodiments, the entry conduit can have a vertical length of approximately 7 mm. After passing through the entry conduit, the collagen solution comes into contact with the extrusion plate, travels through the channels, and exits to the formation buffer in the vertical tank where the sub-fibers can be formed.

The cross-sectional view of FIG. 8 also depicts the offset disposition of the first spinneret 352 at the bottom of the vertical tank. In other words, it can be seen that one side (e.g., a distal side 858) of the first spinneret 352 is nearer to or is less spaced apart from the outer housing 712—represented here as a second distance D2—than the opposing side (e.g., a proximal side 854), represented as a third distance D3. This is due to the presence of the third tube portion 462, which in this case is pressed against and between the proximal side 854 of the spinneret and the outer housing 712, thereby pushing the spinneret toward the distal side of the vertical tank. It can be appreciated that the internal size or diameter of the tank will be selected in part based on the desired size or diameter of both the spinneret and the attached tubing, where the assembly should fit snugly and securely for stability while allowing for free motion in a vertical direction to reach the bottom of the tank while maintaining its horizontal orientation (extrusion zone facing upwards), and later for the spinneret to be easily retrieved or pulled upward and out of the vertical tank.

As described herein, as the plurality of sub-fibers float upward along the height of the vertical tank, they become stronger and more resilient. Referring now to FIGS. 9A and 9B, an isometric top-down view of an interior 920 of the first horizontal bath 342 is shown. In the magnified view of FIG. 9B, it can be seen that a passage can be formed by the stacking of a panel opening 918 in the mating panel 510 atop the opening 516 at the topmost first end 316 of the first vertical tank 312, which can be continued by the stacking of a bath opening 916 formed in the lower surface of in the first horizontal bath 342. Thus, there can be a single outlet 930 from the vertical tank into the horizontal bath. In some embodiments, formation buffer 512 can be held or stored in the compartment of the first horizontal bath 342, and can be continuous with the formation buffer 512 in the first vertical tank 312.

In FIG. 9B, the resultant sub-fibers 710 have reached the top first end 316 of the first vertical tank 312, and moreover have been collected together along a guide rod 940. In one example, the collected sub-fibers 710 are gathered as one unitary bundle at point 950 and passed along a grooved region 960 before being fed into the horizontal formation buffer bath. In some embodiments, collagen fiber can be wet or damp when collected. In such cases, the sub-fibers may tend to stick to each other if they are allowed to touch, especially during collection. Thus, when they are gathered in a bundle and wound together the sub-fibers naturally adhere to one another, forming a single collagen fiber 910. Any number of sub-fibers may be associated, whether twisted or not, to form a bundle, and bundles may be assembled into larger bundles of multiple fibers. For example, bundles may comprise between 2 sub-fibers and about 10,000 fibers, or between about 4 sub-fibers and about 6,000 fibers, typically between about 8 sub-fibers and about 4,000 fibers, and more typically between about 12 sub-fibers and about 2,000 fibers. Then, bundles may be combined, by twisting or otherwise, to form larger bundles. Bundles that are combined need not have equal numbers of fibers. Bundles may be described by the number of fibers in the bundle. For example, a 5-fiber bundle may be called a penta-fiber; 8 fibers would produce an octa-fiber, and so on. In some embodiments, systems and equipment with other numbers of nozzles or extruders may be used to produce such bundles.

In some embodiments, the bundled collagen fiber 910 can be maintained under tension by a tensioner as the fiber is dehydrated, and/or until it is wound on a collector. Typically, a grooved cylinder or spool can be a suitable collector, particularly for wet fibers. For example, a spool at the end of the system can be rotated at a speed that yields a draw speed of between about 2 times the fiber formation rate and about 4 times the fiber formation rate, typically between about 2.5 times the fiber formation rate and about 3.5 times the fiber formation rate, and more typically between about 2.75 and 3.25 the fiber formation rate. The translational speed may be adjusted to adjust separation between fibers on a spool. In some embodiments, at the end of the polymerization period, collagen fiber has been produced and can be separated from the formation buffer solution.

In embodiments of the disclosure, the bundled fiber can be a biopolymer fiber comprising collagen. Once dried, this biopolymer fiber has one or more of the following characteristics: (a) an ultimate tensile strength of between at least 170 MPa (~3N) to more than 340 MPa (~6 N); (b) a modulus of elasticity of between about 1.4 GPa to about 8.5 GPa; (c) a strain at break of between about 4 percent and about 20 percent elongation; (d) an average fiber diameter between about 110 μm (for a 16-fiber bundle) and about 70 μm; and (e) maintains a strength that is higher than reported in literature even after soaking in biological fluid for more than 30 minutes. The fiber exhibits an ordered, longitudinally-oriented structure, and the fiber allows attachment and infiltration of cellular growth. The manufactured biopolymer collagen fiber can be used to develop implantable biopolymer scaffolds for supporting repair of a soft tissue injury, or for repair or replacement for a human body part, where the scaffold comprises at least one biopolymer sheet comprising biopolymer fibers. The sheet can comprise fibers arranged in a typical way for convenience of handling during use. For example, a single fiber would be exceedingly difficult to use because of the small diameter. Thus, it can be necessary or appropriate to form scaffolds, or structures larger than a single fiber, to provide fiber-containing products suitable for repair or replacement of a body part. Thus, for example, it is possible to braid several fibers together to form a strand comprising collagen fibers. Such a strand may be useful, for example, to oversew a rupture in a ligament or tendon.

In embodiments of the disclosure, atelocollagen and telocollagen may be used to form microfluidics extruded collagen microfibers which then can be crosslinked with biological and benign crosslinkers such as glyoxal or DL-Glyceraldehyde (DLG). These cross-linked fibers demonstrated hydrated ultimate tensile strength near 300 MPa and modulus over 3 GPa, significantly stronger than 50 other crosslinking strategies tested and exceeding native human Achilles tendon and anterior cruciate ligament strength. Glyoxal cross-linked fibers further retained 50% of the initial load-bearing capacity through 3-6 months in culture. Collagen fibers implanted in rats demonstrated biocompatibility, promoted the production of new, host-generated aligned collagen growing along the fibers, and in the case of glyoxal crosslinking, promoted an elevated pro-regenerative M2 macrophage response. Embodiments of the disclosure demonstrate marked improvements in healing compared with other crosslinked fibers, making embodiments of the disclosure superior fibers for generating strong collagen sutures or use as a device for ligament, tendon, or other soft tissue repairs

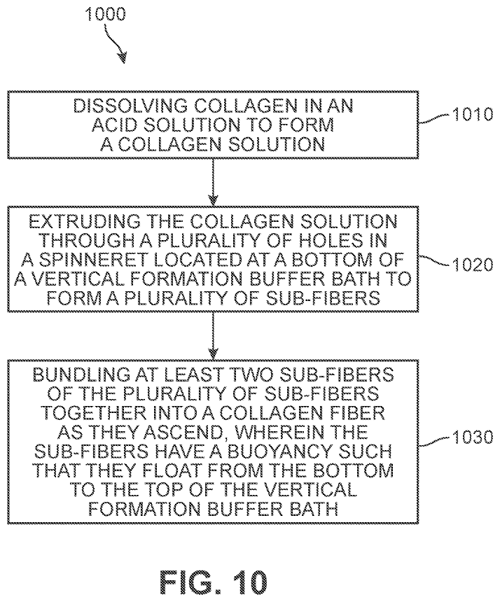

FIG. 10 is a flow chart illustrating an embodiment of a method 1000 of producing a collagen biopolymer fiber. The method 1000 includes a first step 1010 of dissolving (generally solid) collagen in an acid solution to form a collagen solution, and a second step 1020 of extruding the collagen solution through a plurality of holes in a spinneret located at a bottom of a vertical formation buffer bath to form a plurality of sub-fibers. The method 1000 also includes a third step 1030 of bundling (e.g., through a winding process) at least two sub-fibers of the plurality of sub-fibers together into a collagen fiber as the at least two sub-fibers ascend the vertical formation buffer bath. Furthermore, the plurality of sub-fibers and the resulting collagen fiber have a buoyancy such that they float from the bottom of the vertical formation buffer bath when extruded to the top of the vertical formation buffer bath.

In different embodiments, the method may include additional steps or aspects. For example, in some embodiments, the method 1000 also includes passing the collagen solution through a flow meter device that is in communication with a pressure controller, thereby maintaining a substantially constant flow rate. In another example, the method 1000 includes steps of connecting a first end of a tube from a container in which the collagen solution is disposed, and connecting a second end of the tube to the spinneret, thereby providing fluid communication between the collagen solution and the spinneret. In some embodiments, the method 1000 also includes connecting the second end of the tube to an entry port on the spinneret, the entry port providing access to a first hole of the plurality of holes, such that the collagen solution passes out of the second end of the tube and into a first opening of the first hole, upward through a channel, and is extruded out of a second opening of the first hole as a first filament. In some embodiments, the second opening is directly above the first opening with respect to a vertical axis. In another example, the method 1000 includes forming a sheath around the first filament as the first filament is extruded, thereby forming a first sub-fiber that floats upward from the bottom of the vertical formation buffer bath to a top of the vertical formation buffer bath. In one embodiment, the method 1000 further includes extruding one filament through each hole of the plurality of holes in the spinneret. In another example, each hole of the plurality of holes corresponds to a substantially vertical channel that extends between a first opening formed along a lower portion to a second opening formed along an upper portion, wherein the lower portion is encased within the spinneret and the upper portion is disposed on an exterior surface of the spinneret.

Other methods may be contemplated within the scope of the present disclosure. For example, in some embodiments, a method of producing a collagen biopolymer fiber includes a first step of connecting, via one or more tubes, a first container of collagen solution to a spinneret, a second step of inserting the spinneret into a top opening of a vertical formation buffer bath, a third step of releasing the spinneret into the vertical formation buffer bath, a fourth step of extruding the collagen solution through a plurality of holes in the spinneret as the spinneret descends from the top opening to a bottom of the vertical formation buffer bath, and a fifth step of forming a plurality of sub-fibers.

In such embodiments, the method may include additional steps or aspects. In some embodiments, the method also includes a step of dissolving collagen in an acid solution to form the collagen solution. In another example, the method includes bundling or gathering at least two sub-fibers of the plurality of sub-fibers together into a collagen fiber. In some embodiments, each of the plurality of sub-fibers have a buoyancy (relative to the surrounding formation buffer bath liquid) such that they float and ascend from the bottom of the vertical formation buffer bath toward the top opening after extrusion from the spinneret. In another example, the method includes passing the collagen solution through a flow meter device that is in communication with a pressure controller, thereby maintaining a substantially constant flow rate of the collagen solution to the spinneret. In one embodiment, the method further includes pulling the collagen fiber out of the vertical formation buffer bath via the top opening, and passing the collagen fiber through a horizontal formation buffer bath. In some embodiments, the collagen fiber includes a first sub-fiber and a second sub-fiber, and the method further includes determining the first sub-fiber and the second sub-fiber have separated while being pulled out of the vertical formation buffer bath, and re-joining the first sub-fiber and the second sub-fiber as they pass through the horizontal formation buffer bath.

As described herein, some of the proposed embodiments can be understood to include an apparatus for producing a collagen biopolymer fiber. The apparatus can include a vertical formation buffer bath (tank) extending from a bottom end to a top opening, a spinneret disposed along the bottom end of the vertical formation bath, a container of acidified collagen solution, and a tubing system connecting the container of collagen solution to the spinneret. In some embodiments, the spinneret includes a plurality of holes, and each hole of the plurality of holes corresponds to a substantially vertical channel that extends between a first opening formed within a body portion of the spinneret to a second opening formed along an exterior surface of the spinneret. In another example, the apparatus also includes a flow meter and a pressure controller. In one embodiment, the apparatus also includes a horizontal formation buffer bath that is in fluid communication with and disposed orthogonally relative to the vertical formation buffer bath. In some embodiments, the spinneret is sized and dimensioned so as to fit snugly into a top opening of the vertical formation buffer bath and descend to the bottom of the vertical formation buffer bath.

While various embodiments are described, the description is intended to be exemplary, rather than limiting and it will be apparent to those of ordinary skill in the art that many more embodiments and implementations are possible that are within the scope of the disclosed embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted. Further, unless otherwise specified, any step in a method or function of a system may take place in any relative order in relation to any other step described herein.

What is claimed is:

1. A method of producing a collagen biopolymer fiber, comprising the steps of:
dissolving collagen in an acid solution to form a collagen solution;
extruding the collagen solution through a plurality of holes in a spinneret located at a bottom of a vertical formation buffer bath to form a plurality of sub-fibers;
bundling at least two sub-fibers of the plurality of sub-fibers together into a collagen fiber as the at least two sub-fibers ascend the vertical formation buffer bath; and
wherein the plurality of sub-fibers and the collagen fiber have a buoyancy such that they float from the bottom of the vertical formation buffer bath when extruded to a top of the vertical formation buffer bath.

2. The method of claim 1, further comprising passing the collagen solution through a flow meter device that is in communication with a pressure controller, thereby maintaining a substantially constant flow rate.

3. The method of claim 1, further comprising:
connecting a first end of a tube to a container in which the collagen solution is disposed; and
connecting a second end of the tube to the spinneret, thereby providing a path for the collagen solution to flow from the container into the spinneret.

4. The method of claim 3, further comprising connecting the second end of the tube to an entry port on the spinneret, the entry port providing access to a first hole of the plurality of holes, such that the collagen solution passes out of the second end of the tube and into a first opening of the first hole, upward through a channel, and is extruded out of a second opening of the first hole as a first filament.

5. The method of claim 4, wherein the second opening is directly above the first opening with respect to a vertical axis.

6. The method of claim 4, further comprising:
forming a sheath around the first filament as the first filament is extruded, thereby forming a first sub-fiber that floats upward from the bottom of the vertical formation buffer bath to a top of the vertical formation buffer bath.

7. The method of claim 1, further comprising extruding one filament through each hole of the plurality of holes in the spinneret.

8. The method of claim 1, wherein each hole of the plurality of holes corresponds to a substantially vertical channel that extends between a first opening formed along a lower portion to a second opening formed along an upper portion, the lower portion being encased within the spinneret and the upper portion disposed on an exterior surface of the spinneret.

9. A method of producing a collagen biopolymer fiber, comprising the steps of:
connecting, via one or more tubes, a first container of collagen solution to a spinneret;
inserting the spinneret into a top opening of a vertical formation buffer bath;
releasing the spinneret into the vertical formation buffer bath;
extruding the collagen solution through a plurality of holes in the spinneret as the spinneret descends from the top opening to a bottom of the vertical formation buffer bath; and
forming a plurality of sub-fibers.

10. The method of claim 9, further comprising dissolving collagen in an acid solution to form the collagen solution.

11. The method of claim 9, further comprising bundling at least two sub-fibers of the plurality of sub-fibers together into a collagen fiber.

12. The method of claim 9, wherein each of the plurality of sub-fibers have a buoyancy such that they float and ascend from the bottom of the vertical formation buffer bath toward the top opening after extrusion from the spinneret.

13. The method of claim 9, further comprising passing the collagen solution through a flow meter device that is in communication with a pressure controller, thereby maintaining a substantially constant flow rate of the collagen solution to the spinneret.

14. The method of claim 11, further comprising:
pulling the collagen fiber out of the vertical formation buffer bath via the top opening; and
passing the collagen fiber through a horizontal formation buffer bath.

15. The method of claim 14, wherein the collagen fiber includes a first sub-fiber and a second sub-fiber, and the method further comprises:
determining the first sub-fiber and the second sub-fiber have separated while being pulled out of the vertical formation buffer bath;
re-joining the first sub-fiber and the second sub-fiber as they pass through the horizontal formation buffer bath.

16. An apparatus for producing a collagen biopolymer fiber, the apparatus comprising:
a vertical formation buffer bath extending from a bottom end to a top opening;
a spinneret disposed along the bottom end of the vertical formation buffer bath;
a container of collagen solution; and
a tubing system connecting the container of collagen solution to the spinneret.

17. The apparatus of claim 16, wherein the spinneret includes a plurality of holes, and each hole of the plurality of holes corresponds to a substantially vertical channel that extends between a first opening formed within a body portion of the spinneret to a second opening formed along an exterior surface of the spinneret.

18. The apparatus of claim 16, further comprising:
a flow meter; and
a pressure controller.

19. The apparatus of claim 16, further comprising a horizontal formation buffer bath that is in fluid communication with and disposed orthogonally relative to the vertical formation buffer bath.

20. The apparatus of claim 16, wherein the spinneret is sized and dimensioned so as to fit snugly into a top opening of the vertical formation buffer bath and descend to the bottom end of the vertical formation buffer bath.
